(19) United States
(12) Patent Application Publication
(10) Pub. No.: US 2017/0367391 A1
(43) Pub. Date: Dec. 28, 2017

(54) Bars and Shakes utilizing proprietary blend of herbs for enhanced human lactation and breastfeeding.

(71) Applicant: Wendy Colson, Carlsbad, CA (US)

(72) Inventor: Wendy Colson, Carlsbad, CA (US)

(21) Appl. No.: 15/351,004

(22) Filed: Nov. 14, 2016

Related U.S. Application Data
(60) Provisional application No. 62/255,447, filed on Nov. 14, 2015.

(51) Int. Cl. A23L 33/105; A23L 33/16; A61K 36/48; A23L 33/115; A23L 33/125; A23L 33/185; A61K 36/896; A23P 10/35

(52) U.S. Cl. CPC A23L 33/105; A23L 33/185; A23L 33/16; A61K 36/896; A61K 36/48; A23L 33/115; A23L 33/125; A23P 10/35; A61K 2300/00

(57) ABSTRACT

This invention is directed toward a bar and a shake that contain a blend of herbs for enhanced human and animal lactation and breastfeeding.

### CROSS REFERENCE TO RELATED APPLICATIONS

This application claims priority back to U.S. Provisional Application No. 62/255,447, filed Nov. 14, 2105, a copy of which is attached to this application and the contents of which are incorporated by reference.

### STATEMENT REGARDING FEDERALLY SPONSORED RESEARCH OR DEVELOPMENT

This invention was not federally sponsored.

### INVENTOR

Wendy Colson, citizen of US, resident of US.

### FIELD OF THE INVENTION

This invention relates to human lactation and breastfeeding, and products that will enhance the same. Lactation (definition): The secretion or formation of milk by the mammary glands. And more specifically, to increase or maintain milk secretion during the lactation period using a blend of ingredients known in lactation as a galactagogue. Galactagogue (definition): is a substance (i.e. food, herbs, medicines) that promotes lactation in humans and other mammals. The invention described in this patent application includes the addition of a proprietary blend of lactation supporting herbs (galactagogues) which are custom blended and added as a single ingredient into a convenient baked food-bar or protein-type shake powder.

Both food items are meant to be ingested by a lactating woman for the purpose of increasing, maintaining, and supporting her breastmilk supply. In blending these herbs so that the consumer would only have to consume ONE bar or shake a day to raise the prolactin (the hormone responsible for milk production) levels, the invention avoids the need for a woman to purchase and consume a variety of products. By using a blend of herbs as opposed to a single herb, the invention increases the likelihood that one or more of the herbs will be effective for the lactating woman who is the consumer.

The proprietary blend of milk-boosting herbs (Galactagogues) includes moringa leaf, shatavari root, fenugreek, turmeric, and cinnamon (listed on packaging as spice per FDA packaging disclosure guidelines). The same herbal blend is added to both the bar and shake product and is made ahead and added to both products as a single ingredient. Each herb is measured by weighing the amount needed which has been derived from trial and error under the oversight of a board certified lactation consultant (IBCLC) and registered nurse. The finished combination of herbs blended together gets added to the bar product, called Boobie Bars™ in the industry.

To make the Boobie Bars, the process starts with heating coconut oil just enough to liquefy it, then add the flaxseed meal (Galactagogue) and proprietary blend of 5-6 herbs listed above. Again, the blend is added as a single ingredient. Next, the cane sugar is added and the ingredients are lightly mixed before adding the remaining dry ingredients which include flour, baking soda, salt, an oat mixture (Galactagogue) which consists of half whole oats and half cut oats. The final ingredients added to the mixture define what flavor the final bar product becomes. Currently, the three Boobie Bar flavors are Oatmeal Chocolate Chip, Blueberry Coconut, and Gluten-free Peanut Butter. The final ingredients depend on the flavor bars being made and include chocolate chips (Oatmeal Chocolate Chip), dried blueberries and coconut (Blueberry Coconut), or natural peanut butter (Gluten-free Peanut Butter). The final mixture is then mixed and put through an extruder device which squeezes the blend and it's ingredients under extreme pressure compacting them into a tight dough to form the bars. The co-packer's machinery cuts the bars into specific sizes/weight, flash baking in commercial ovens, cooling, then packaging.

To make the shake version, which I call Boobie Shake, the invention uses vegan protein powder which is currently pea protein and use it as the base. Then, the remaining ingredients are added, including the proprietary blend of 5 herbs are added to get the optimal results for lactating woman to aid in breastmilk supply based on the advice of a board certified lactation consultant (IBCLC) and registered nurse.

Currently, the remaining ingredients added to the vegan protein base and herbal proprietary blend are cacao powder, flax, chia seeds, maca powder, sugar, stevia, lecithin, and a blend of probiotic powders. All the ingredients are mixed and measured into packaging to sell. The shake's consumer then chooses the liquid of their choice (water, milk, non-dairy milk alternative, etc.) and adds a recommended measured amount of the shake's ingredients (currently 20-30 grams) and mixes before ingesting. Both the bars and shake mixture are 100% vegan and kosher. In addition to being vegan and kosher, Boobie Shake and the Peanut Butter flavored Boobie Bar are 100% gluten-free. These exclusions of allergens such as dairy, eggs, and gluten were done “on purpose” to both products because my education, training, and experience as board certified lactation consultant tells me what a lactating woman ingests can pass through her breastmilk causing problems to her baby's immature digestive tract. Therefore, the majority of lactating moms prefer to avoid dairy and gluten.

History of the breastfeeding-enhancement industry. The most well-known ways to ingest galactagogues prior to this invention of Boobie Bars and Boobie Shake were “mother's formula” teas and lactation cookies. There are several tea manufacturers who formulate all kinds of tea blends and have included a “mother's” tea to their line of products. Some of the best known commercial “mother's” teas companies are Traditional Medicinals and Earth Mama Angel Baby. These companies commonly use a blend of teas leafs, the only tea leaf which matches just one of this invention's proprietary blend's ingredients is fenugreek tea leafs. Boobie Bar uses ground fenugreek powder not leafs. The problem with ingesting teas for the purpose of promoting lactation is that any intent to promote a healthy milk supply is being diluted with 6-8 ounces of water. Therefore, the percentage of galactagogue being ingested is minimal requiring large amounts of tea to be consumed to be effective.

Lactation Cookies are more recently becoming popular. This started out a few years ago as simple oatmeal (Galactagogue) cookie recipes found online with added known Galactagogues like flaxseeds. None of the cookie recipes are evidenced based. Therefore, in the medical field of breastfeeding medicine, lactation professionals do not recommend lactation cookies as an effective way to promote a healthy lactation. Then the first commercially made lactation cookies emerged in 2009.

The two commercial companies are Milkmakers and Milkin' Cookies. Again, they are a basic oatmeal cookie recipe with the only Galactagogues being oats and flax. Both food ingredients which are in a Boobie Bar. Neither product/manufacture has added a blend of herbs (Galactagogues) with the exception of Milkmakers which adds brewer's yeast only as a single herb not blend.

The commercial cookies have no clinical studies done by either company which would validate their effectiveness in promoting lactation. In contrast, three of the herbs in my invention, the blend of 5-6 herbs have been studied and published in lactation journals as commonly used galactogogues. These three herbs include moringa, shatavari, and fenugreek. Another component which makes the lactation cookies marketed to lactation women a poor choice is both companies do not have a gluten free prepared option and neither company has vegan products eliminating the common allergens which can upset a baby's immature digestive tract. Similar to a Boobie Bar, the cookies have about 210-230 calories each. However, unlike a Boobie bar the recommended dose for the cookies is 2-3 a day. That adds up to over 600 calories per day for a snack in a population who is looking to lose weight gained in pregnancy not gain.

Neither the tea's nor the cookie's formulas were created by a board certified lactation consultant (IBCLC). IBCLC stands for Internationally Board Certified Lactation Consultant. These initials are the highest initials you can obtain in terms of lactation education. Even higher than the initials MD. Internationally board certified means that a holder of such a certificate can practice lactation on any continent.

Human lactation and breastfeeding is the biological norm for the feeding of human mammals. Lactation and receiving mother's human milk offers both the woman and child many health advantages both while actively lactating and for the prevention of childhood and adult diseases. Therefore, breastfeeding and/or pumping and giving breastmilk via a bottle or other feeding method is considered preventable health. However, the most recent CDC Breastfeeding Report Card states that 79% of newborn infants started to breastfeed, yet only 27% were still breastfeeding at age one, falling short of the minimum recommended age by world and local governmental agencies. Problem: Low milk supply is a major reason why many moms do not reach their breastfeeding goals.

World and local government agencies recommendations on breastfeeding:

World Health Organization (WHO) recommendations on breastfeeding: Exclusive breastfeeding is recommended up to 6 months of age, with continued breastfeeding along with appropriate complementary foods up to two years of age.

American Academy of Pediatric recommendation on breastfeeding: The AAP recommends that babies be exclusively breastfed for about the first 6 months of life. Babies should continue to breastfeed for a year and for as long as is mutually desired by the mother and baby

Surgeon General, Call to Action, 2011: For nearly all infants, breastfeeding is the best source of infant nutrition and immunologic protection, and provides remarkable health benefits to mothers as well (2011, Surgeon General, Call to Action).

Implementing the Patient Protection and Affordable Care Act of 2010 (ACA) Mandatory Insurance Coverage: breastfeeding insurance coverage applies to all health plans except grandfathered plans. Under the law, 23 preventive health services for women are to be covered with no copayment, co-insurance or deductible in non-grandfathered plans.

Solution: The invention Boobie Bars and Boobie Shake with the added blend of milk-promoting galactagogues, aka herbs, provides a convenient single bar or shake drink as a way to ingest common galactogogues which have been studied in the field of breastfeeding medicine to help increase and maintain breastmilk supply. Therefore, decreasing a major reason a lot of women cite for prematurely weaning (quitting) lactating before the recommended time periods.

The prior has several examples of attempts to resolve this problem the existing food lactation products currently on the market fall short of being effective products for supporting lactating women's milk supply for the following reasons:

Tea is a very weak form of herbs. This is because the herbs are being diluted in 6-8 cups of water to prepare as directed. A lactating mom would have to drink large amounts (quarts) to ingest a decent amount to raise her blood levels of prolactin, the hormone responsible for making breastmilk.

Lactation teas currently on the market only contain one herb (in tea leaf form) that is also included in my blend. That herb is fenugreek. My blend uses ground fenugreek compared to the tea which just uses the leaf to be diluted in hot water. Fenugreek is usually the herb in the highest concentration found in lactation teas. However, high levels of fenugreek are contraindicated in mothers who have low blood sugar or hypothyroidism. This warning would give mothers who suffer from these conditions limited options when looking for a galactagogue food product currently being sold on the market decreasing their success in boosting their milk supply if choosing lactation teas. My blends highest concentrated herbs are moringa followed by shatavari

Lactation cookies—since 2009, there are only two commercial lactation cookie products on the market. The lactation cookies are missing vital components which would make them a good option for a lactating mom whose goal is to ingest for the purpose of increasing or maintain her breastmilk supply.

The lactation cookies do not contain any herbal blends and only one of the two manufacture's products contain any herb.

The effectiveness of the cookies is anecdotal and lacks evidence for their promotion of lactation. Neither two lactation companies have performed any studies to back up their claims. The following herbs in my blend moringa, shatavari, and turmeric have been studied in the field of lactation and other medicines. The cookie recipe parallels a basic oatmeal cookie made with butter, eggs, and flours which contain gluten.

No vegan lactation cookie is available and no gluten-free option is available in the already prepared versions of lactation cookies. Again, as previously mentioned. The most common allergens which should be eliminated if a lactating mom's baby's tummy is sensitive is dairy, eggs, soy, and gluten.

The recommended “dose” is to ingest 2-3 cookies. This adds a lot of unnecessary calories to a nursing diet. Again, this “dose” is anecdotal.

Thus there has existed a long-felt need for a product that provides the addition of a proprietary blend of lactation supporting herbs (galactagogues) which is custom blended adding them as a single ingredient into a convenient baked food-bar or protein-type shake powder to use in place of the lactation teas and lactation cookies which are already on the commercial market. The product described in this patent application is the first of its kind. There is no other way to ingest lactation herbs currently except for teas and pills (nutritional supplements). The closest food item marketed to lactating women is the cookies, but they do not contain any herbs. Both products described in this patent application are convenience foods which are designed with the busy mother in mind. She can grab and go. No hot water needed, no watching the clock for the tea to brew and this mom doesn't need to consume 2-3. This invention added a proprietary blend of milk-boosting herbs to very popular convenience foods, a “protein-type” bar and a “protein” powder to make shakes with. Because the bar is vegan, laboratory tests showed a low risk for mold spore growth giving it a long shelf-life, making it even more convenient. Both the bar and shake food are meant to be ingested by a lactating woman for the purpose of increasing, maintaining, and supporting her breastmilk supply. The invention blends these herbs so that the consumer would only have to consume ONE bar or ONE shake a day to raise the serum prolactin levels (the hormone responsible for milk production). By using a blend of herbs, as opposed to a single herb, it increases the likelihood that one or more of the herbs will be effective for the lactating woman who is the consumer.

The current invention provides just such a solution by having a bar and shake that combine a number of potentially useful herbs into a single product that can be consumed by a breastfeeding woman. The core of what makes this product stand out in this industry is that both the bar and the shake contain herbs known as galactagogues. The invention includes the addition of a proprietary blend of lactation supporting herbs (galactagogues) which has never been. By using a blend of herbs as opposed to a single herb, the invention increases the likelihood that one or more of the herbs will be effective for the lactating woman who is the consumer.

The proprietary blend of milk-boosting herbs (Galactagogues) were chosen for each one of its known health benefits and my personal success with my patients as a board certified lactation consultant (IBCLC), the gold standard in lactation education which includes moringa leaf, shatavari root, fenugreek, turmeric, and cinnamon (listed on packaging as spice per FDA packaging disclosure guidelines). The benefits of each herb are listed here.

Morinqa—Often referred to as “the tree of life” because of its medicinal and nutritional benefits over the span of many centuries. The leaves of the moringa plant have been used to increase breastmilk supply for generations in the Philippines and throughout India and Asia.

Shatavari Ro(}t (Asparagus racemosus): The most widely used galactagogue (milk-making herb) in India used to improve the production of breastmilk in nursing moms. Studies have shown an increase in prolactin levels, the hormone responsible for making milk.

Currently the shake is 100% gluten-free and one out of three of the bar flavors is. Gluten free foods sales were reported back in a 2014 to have surged close to 70%. The invention provides a gluten free flavor, peanut butter, is not the best seller, but is the number two seller with a high reorder rate.

Making a baked bar which is gluten free using special flours is more costly to make but if the demand continues to drive this market, it will likely follow by making all Boobie Products gluten free since the population is becoming sensitive to wheat, gluten and reporting GI upset therefore avoiding gluten made products. In lactation, gluten is being eliminated when removing other more known allergens like dairy and soy do not rectify the problem of a fussy baby who is experiencing GI upset from possible transfer of allergens in mother's milk.

Fenugreek (seed): Fenugreek is a natural product that is a member of the pea family. Reports of the successful use of fenugreek as a galactagogue have been documented as far back as 1945 making it the best known and most widely use galactagogue. When taking as a nutritional supplement in pill form, each pill usually contains 610 mg and requires 2-3 pills three times a day. These high doses found in nutritional supplements, aka pills can decrease blood sugar and are not recommended for hypothyroid and diabetic lactating patients.

Turmeric: Many women are unable to breastfeed their baby properly due to clogged mammary glands and swollen breasts, which are quite painful. Turmeric is a known anti-inflammatory of the breast tissue. It's been noted that woman should consume turmeric daily for breast health and prevent breast cancer. In fact, this herb is the best known remedy for all inflamed tissues in your body.

Cinnamon: labeled as spice on packaging per FDA disclosure guidelines). Ayurveda, the ancient healing system of India, often uses cinnamon to stimulate circulation as well as to increase the bio-availability of other herbs In Chinese medicine, cinnamon is used to improve circulation. I chose this herb to increase the “flow” of milk known as the milk ejection reflex.

### Objects of the Invention

It is therefore an object of the present invention to provide a proprietary blend is put into a baked bar product and a shake powder mix.

Other objects of the invention include:

Convert all Boobie Bars and future products to be 100% gluten free. I currently use in the Gluten-free Peanut Butter Boobie Bar and Boobie Shake. Gluten free foods sales were reported back in a 2014 to have surged close to 70%. My current gluten free flavor, peanut butter, is not my best seller, but is my number two seller with a high reorder rate. Making a baked bar which is gluten free using special flours is more costly to make but if the demand continues to drive this market, I will likely follow by making all Boobie Products gluten free since the population is becoming sensitive to wheat and gluten, reporting GI upset therefore, avoiding gluten made products. In lactation, gluten is being eliminated from a mother's diet when removing other more known allergens like dairy and soy do not rectify the problem of a fussy baby who is experiencing GI upset from possible transfer of allergens in mother's milk. Prepared protein-type health shakes/drinks which do not require any mixing.

Covert oil in bar to half palm or other oil instead of being 100% coconut oil. This would be for cost savings, production reasons, and the demand for coconut oil keeps rising which worries me about the availability. Before launching our production was put on hold because a typhoon hit the Philippines where our coconut oil was coming from and delayed our order with the co-packer. Having a more sustainable oil in the recipe is safer to keep up with the demands of inventory.

Currently, the Boobie shake powder is chocolate but adding a vanilla flavored one would be a goal.

Single serving oatmeal which just requires the addition of hot water. Since oats are a natural galactagogue and single serving oatmeal cups and packets are popular right now.

Nut butters—single serving nut butters are a growing market. There is natural peanut butter pouches, almond butter, and even chocolate added to nut butter varieties on the market.

Granola/trail mixes—again, convenient food pouches to grab and go that contain the blend of herbs.

Adding additional Boobie Bar flavors when demanded—brownie flavor, spice such as pumpkin or molasses, peanut butter chocolate chip. Additional embodiments include pet treats to enhance low milk supply in dogs and cats, along with other mammals.

### Statement of the Invention

Accordingly, one aspect the invention includes a Six herb blend mix list of ingredients:

1 Moringa leaf

2 Shatavari root

3 Fenugreek

4 Turmeric

5 Cinnamon

Five Herb GLUTEN FREE Mix of Ingredients:
1. Moringa leaf
2. Shatavari root
1. Fenugreek
2. Turmeric
3. Cinnamon

Current Three Flavors of Boobie Bars

1. Oatmeal Chocolate Chip

2. Blueberry Coconut

3. Gluten-free Peanut Butter

Different Suggested Ways to Eat a Boobie Bar to Get Your Daily Intake of Herbs to Raise Blood Prolactin Levels

1. Bar form by itself

2. Crumble bar on Greek yogurt, oatmeal, or ice cream

3. Blend bar in a smoothie to

Boobie Shake Ingredients-Current

1. Raw cacao

2. Vegan protein powder, currently pea but have used brown rice powder also

3. Chia seeds

4. Maca root powder

5. Flaxseed meal

6. Soy or sunflower lecithin

7. Five blend of herbs listed above

8. Probiotic powders

9. Sugar-evaporated cane

10. Stevia or alternative sweeteners

The way the bars are made-current
1. Made by a Co-packer-After all ingredients have been mixed, they are put through an extruder device, compacting them and forming their “bar” shape. Then cut for specific weight (grams)/serving size. Then flash baked, not too much oven time so the herbs are kept potent. It's a vegan bar so they are not baked to kill bacteria from eggs etc. Baked just enough to harden dough and bake off the flour taste.
2. The way the bars are made-past
   Made homemade in my home kitchen in small production. Same steps as above but they were hand formed then baked. Weighed after baking and cut excess off to make them look uniform and share similar weights.

Current List of Ingredients—Oatmeal Chocolate Chip
whole wheat flour, cane sugar, whole grain oats, coconut oil, brown cane sugar, chocolate chips, proprietary herb blend, flaxseed meal, cocoa powder, natural flavor (vanilla), baking soda, and kosher salt

Current List of Ingredients—Blueberry Coconut
whole wheat flour, cane sugar, whole grain oats, coconut oil, brown cane sugar, dried blueberries, proprietary herb blend, unsweetened dried coconut, flaxseed meal, natural flavor (blueberry and vanilla), baking soda, and kosher salt

Current List of Ingredients—Gluten Free Peanut Butter
All natural peanut butter, cane sugar, gluten free oats, brown cane sugar, coconut oil, coconut flour, proprietary herb blend, flaxseed meal, natural flavor (vanilla), baking soda, and kosher salt

### BRIEF DESCRIPTION OF THE FIGURES

One preferred form of the invention will now be described with reference to the accompanying drawings.

FIGS. 1 and 2 are flow charts of how the invention is made.

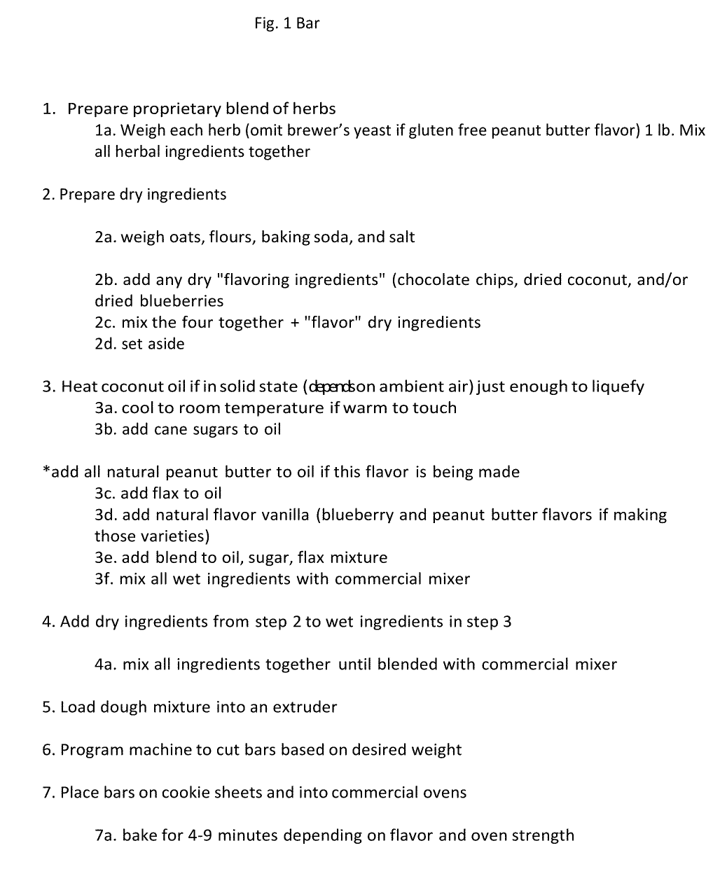

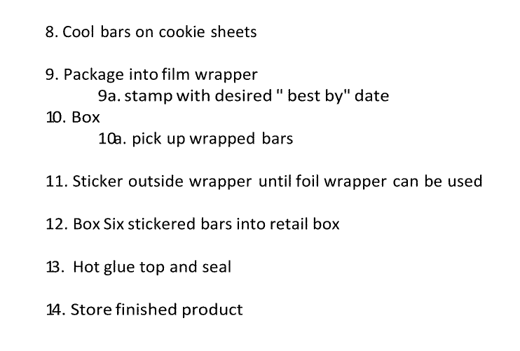

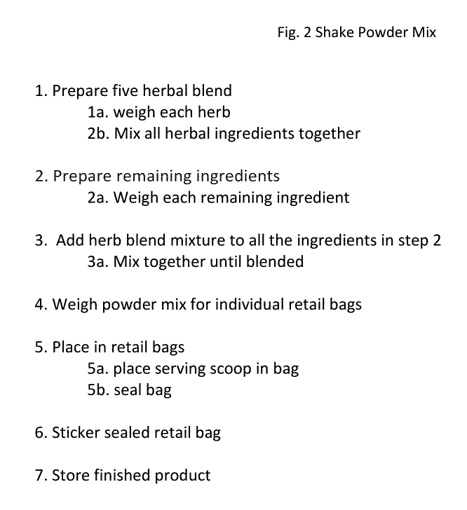

### DETAILED DESCRIPTION OF THE FIGURES

The flow charts illustrate each step of the process by which the bars and shakes are made.

That which is claimed:

1. An edible bar for the promotion of human lactation comprising moringa leaves, shatavari root, ground fenugreek seed, turmeric and cinnamon, where the edible bar comprises a greater concentration of moringa leaves than shatavari root, where the edible bar comprises a greater concentration of shatavari root than ground fenugreek seed, turmeric, or cinnamon.

2. The edible bar of claim 1, wherein the edible bar further comprises oats and flax seed.

3. The edible bar of claim 2, wherein the edible bar further comprises flour, coconut oil, salt and sodium bicarbonate.

4. The edible bar of claim 3, wherein the edible bar further comprises cane sugar and vanilla.

5. The edible bar of claim 4, wherein the edible bar further comprises cocoa powder.

6. An edible powder for use in shakes for the promotion of human lactation comprising moringa leaves, shatavari root, ground fenugreek seed, turmeric and cinnamon, where the edible powder comprises a greater concentration of moringa leaves than shatavari root, where the edible powder comprises a greater concentration of shatavari root than ground fenugreek seed, turmeric, or cinnamon.

7. The edible powder of claim 6, wherein the edible powder further comprises vegan protein powder.

8. The edible powder of claim 7, wherein the vegan protein powder comprises pea.

9. The edible powder of claim 7, wherein the vegan protein powder comprises brown rice.

10. The edible powder of claim 6, wherein the edible powder further comprises chia seeds, maca root powder, flax seed, and probiotic powders.

11. The edible powder of claim 6, wherein the edible powder further comprises lecithin.

12. The edible powder of claim 11, wherein the lecithin is soy lecithin.

13. The edible powder of claim 11, wherein the lecithin is sunflower lecithin.

14. A method of manufacturing edible bars comprising the steps of
heating coconut oil, where the coconut oil is heated until it liquefies;
adding dry ingredients, where the dry ingredients comprise moringa leaves, shatavari root, ground fenugreek seed, turmeric and cinnamon together, where there is a greater amount of moringa leaves than shatavari root, where the is a greater amount of shatavari root than ground fenugreek seed, turmeric or cinnamon,
mixing the coconut oil with the dry ingredients;
adding additional ingredients, where the additional ingredients comprise sodium bicarbonate and salt;
mixing the additional ingredients with the dry ingredients and coconut oil to form a final mixture; and
extruding the final mixture through an extruder device.

15. The method of claim 14, further comprising the step cutting the extruded final mixture into bars.

16. The method of claim 15, further comprising the step of baking the bars, where the bars are baked for four to nine minutes.

17. The method of claim 16, further comprising the step of packaging the bars, where each bar is individually packaged.

18. The method of claim 14, wherein the dry ingredients further comprise cane sugar.

19. The method of claim 14, wherein the additional ingredients further comprise flour and oats.

20. The method of claim 14, wherein the additional ingredients further comprise flax seed.
